(19) United States
(12) Patent Application Publication
(10) Pub. No.: US 2008/0208061 A1
(43) Pub. Date: Aug. 28, 2008

(54) Methods and systems for spatial compounding in a handheld ultrasound device

(75) Inventor: Nahi Halmann, Milwaukee, WI (US)

Correspondence Address: DEAN D. SMALL;THE SMALL PATENT LAW GROUP LLP, 225 S. MERAMEC, STE. 725T, ST. LOUIS, MO, 63105, US

(73) Assignee: General Electric Company

(21) Appl. No.: 11/710,773

(22) Filed: Feb. 23, 2007

(51) Int. Cl. A61B 8/00

(52) U.S. Cl. 600/459; 600/437

(57) ABSTRACT

Method and systems for medical ultrasound imaging using a handheld ultrasound imaging system is provided. The ultrasound system includes a probe configured to acquire scan data from an object and a handheld device configured to process the received scan data and perform image compounding.

### BACKGROUND OF THE INVENTION

This invention relates generally to ultrasound systems, and more particularly, handheld and hand-carried ultrasound (or other medical imaging) systems.

Ultrasound imaging is a medical imaging technique for imaging organs and soft tissues in a human body. Ultrasound imaging uses real time, noninvasive high frequency sound waves to produce a two-dimensional (2D) image. Although, ultrasound imaging provides less anatomical information compared to CT or MRI, ultrasound imaging has several advantages in that patients are not exposed to radiation, studies of moving structures may be provided in real-time, the image scan is quickly performed and is inexpensive.

In conventional ultrasound imaging, the image is acquired by a series of parallel scan lines. This results in an image in which some anatomical structures may be “shadowed” by objects closer to the transducer and diagonal structures may not be optimally imaged. Typically, when the boundaries of anatomical structures are parallel to the transducer, the acoustic waves reflect directly back to the transducer with less dispersion and a clear image is obtained. However, diagonal or vertical structures are sub-optimally imaged using conventional ultrasound because of the lower percentage of acoustic energy that reflects back to the transducer. Furthermore, structures that are hidden beneath strong reflectors are also sub-optimally imaged. For instance, a small breast cyst may be hidden behind muscular tissue (e.g., tendons), which is a strong superficial reflector.

In addition, another disadvantage of conventional ultrasound imaging is speckle noise. Speckle noise is a result of interference of scattered echo signals reflected from an object, such as an organ, and appears as a granular grayscale pattern on an image. The speckle noise degrades image quality (e.g., speckles obtained from different angles are incoherent) and increases the difficulty of discriminating fine details in images during diagnostic examinations.

At least some known ultrasound systems are capable of spatially compounding a plurality of ultrasound images of a given target into a compound image. The term “compounding” generally refers to non-coherently combining multiple data sets to create a new single data set. The plurality of data sets may each be obtained from a different steering angle and/or aperture and/or may each be obtained at a different time.

The plurality of data sets or steering frames are combined to generate a single view or compound image by combining the data received from each point in the compound image target that has been received from each steering angle or aperture. Real time spatial compound imaging may be performed by acquiring a series of partially overlapping component image frames from substantially independent steering angles. A transducer array may be utilized to implement electronic beam steering and/or electronic translation of the component frames. The component frames are combined into a compound image by summation, averaging, peak detection, or other combinational means. The compounded image may display relatively lower speckle and better specular reflector delineation than a non-spatially compounded ultrasound image from a single angle.

Handheld or hand-carried ultrasound systems are also known and that provide ultrasound imaging in a more compact and portable unit. In many cases these handheld or hand-carried ultrasound devices are used for interventional procedures in which viewing a needle or a biopsy guide is critical. If the scan lines are only perpendicular to the transducer, a diagonally inserted needle may not visualize well. Thus, these known handheld or hand-carried ultrasound devices may not provide acceptable image quality and thereby can result in possible errors during the procedure.

### BRIEF DESCRIPTION OF THE INVENTION

In an embodiment of the invention, an ultrasound system is provided that includes a probe configured to acquire scan data from an object and a handheld or hand-carried device configured to process the received scan data and perform image compounding. Optionally, a processor is included for processing image data. The processor includes at least one of a data capture module, geometric transformation module, interpolation module, compounding module, battery management module, heat management module, frame processing module, scan conversion module, and resolution selection module. The system performs image compounding of the received data to produce real-time images of the object.

In another embodiment, a method of medical ultrasound imaging using a handheld or hand-carried ultrasound imaging system having a transducer array is provided. The method includes transmitting a plurality of ultrasound waves at a plurality of different angles from the transducers into a region of interest, and receiving ultrasound echoes for each transmitted wave. The received echoes define a plurality of steering frames that correspond to the plurality of different angles. A compound image is produced by combining the plurality of steering frames and displayed on a screen.

In yet another embodiment, a handheld or hand-carried medical ultrasound system is provided that includes a transducer array including a plurality of transducers for transmitting ultrasound signals at a plurality of different angles into a region of interest. The system further includes a receiver for receiving ultrasound echoes for each transmitted ultrasound signal, where each set of received echoes defines a plurality of steering frames corresponding to the plurality of different angles. A signal processor combines the steering frames to produce a compound image that is displayed on a screen.

In still another embodiment, a computer readable medium for use in a handheld or hand-carried medical ultrasound imaging system having an array transducer for transmitting and receiving ultrasound signals into a region of interest is provided. The computer readable medium provides instructions to transmit ultrasound signals at a plurality of different angles into the region of interest. The medium further provides instructions to receive ultrasound echoes for each of the transmitted ultrasound signals. The received ultrasound echoes define a plurality of steering frames that correspond to the plurality of different angles. Furthermore, the medium provides instructions to filter the steering frames using a speckle filter to remove interference of scattered echo signals reflected from the region of interest. In addition instructions to combine a plurality of the filtered steering frames into a compound image and display the compound image are provided.

### BRIEF DESCRIPTION OF THE DRAWINGS

FIG. 1 is a block diagram of an ultrasound system formed in accordance with an embodiment of the present invention.

FIG. 2 is a block diagram of a handheld or hand-carried ultrasound system that utilizes a software backend in accordance with an embodiment of the present invention.

FIG. 3 is a block diagram of a hand-carried or handheld medical imaging device formed in accordance with various embodiments of the invention having a probe or transducer configured to acquire raw medical image data.

FIG. 4 is a pictorial view of a miniaturized ultrasound system in connection with which various embodiments of the invention may be implemented.

FIG. 5 is a plan view of an exemplary pocket-sized ultrasound system in connection with which various embodiments of the invention may be implemented.

FIG. 6 illustrates a sector scan that is performed by scanning a fan-shaped two-dimensional (2D) region in accordance with an embodiment of the invention.

FIG. 7 illustrates an alternative linear scan that is performed by scanning a rectangular 2D region in a direction along an x-axis in accordance with an embodiment of the invention.

FIG. 8 illustrates a convex scan or a curved linear scan that is performed by scanning a partial fan-shaped region in accordance with an embodiment of the invention.

FIG. 9 illustrates an exemplary acquisition of an object acquired by an ultrasound system in accordance with an embodiment of the invention.

FIG. 10 illustrates two sequences for steering transducer elements in accordance with an embodiment of the invention.

FIG. 11 illustrates the acquisition of data samples from a steered frame and a non-steered frame in accordance with an embodiment of the invention.

FIG. 12 is an illustration of spatial compounding in accordance with various embodiments of the invention.

FIG. 13 illustrates the use of a weighting factor for three-angle compounding in accordance with an embodiment of the invention.

FIGS. 14 and 15 illustrate a normal ultra-sound image and an ultrasound image using spatial compounding in accordance with an embodiment of the invention.

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

### DETAILED DESCRIPTION OF THE INVENTION

In the following detailed description, reference is made to the accompanying drawings which form a part hereof, and in which is shown by way of illustration specific embodiments in which the present invention may be practiced. It is to be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

In this document, the terms “a” or “an” are used, to include one or more than one. In this document, the term “or” is used to refer to a nonexclusive or, unless otherwise indicated. In addition, as used herein, the phrase “pixel” also includes embodiments of the present invention where the data is represented by a “voxel”. Thus, both the terms “pixel” and “voxel” may be used interchangeably throughout this document.

Also as used herein, the phrase “reconstructing an image” is not intended to exclude embodiments of the present invention in which data representing an image is generated, but a viewable image is not generated. Therefore, as used herein, the term “image” broadly refers to both viewable images and data representing a viewable image. However, many embodiments generate (or are configured to generate) at least one viewable image.

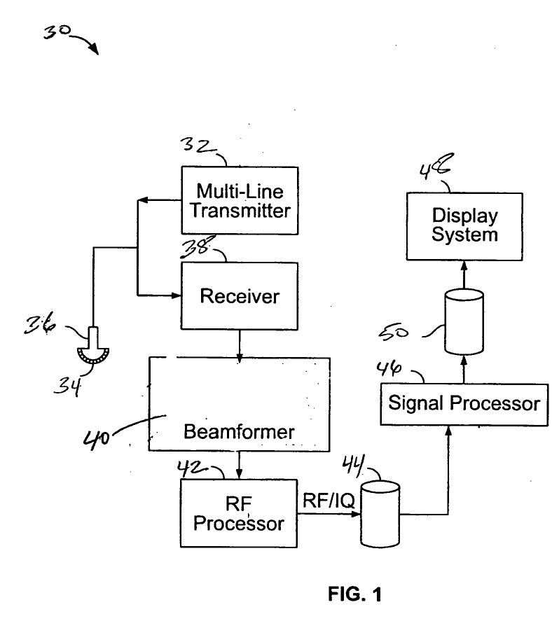

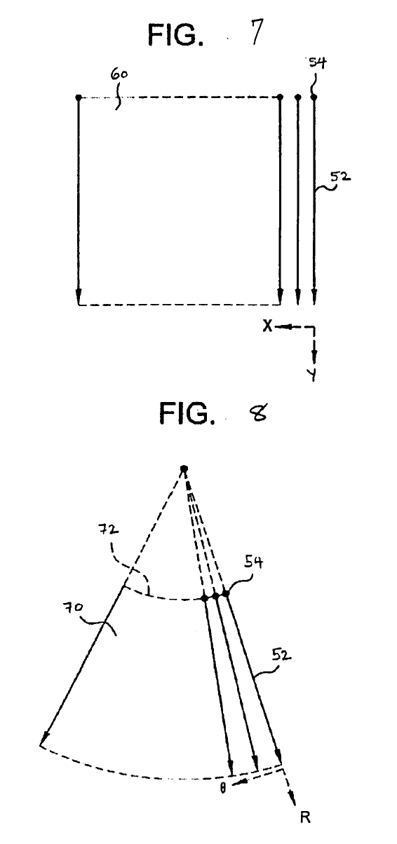

FIG. 1 illustrates a block diagram of an ultrasound system 30 formed in accordance with an embodiment of the present invention. The ultrasound system 30 includes a transmitter 32 that drives transducer elements 34 within a transducer 36 to emit pulsed ultrasonic signals into a body. The transducer elements 34 include piezoelectric elements (not shown) that fire an ultrasound pulse. A variety of geometries for transmitting the ultrasound signals may be used. For instance transducer 36 may be a curved linear probe or a linear probe. The ultrasonic signals are back-scattered from structures in the body, like blood cells or muscular tissue, to produce echoes which return to the transducer elements 34. The echoes are received by a receiver 38, and the received echoes may include undesirable speckle (e.g., interference caused by scattered echo signals reflected from the region of interest). The received echoes are passed through a beamformer 40 that performs beamforming and outputs an RF signal. The RF signal then passes through an RF processor 42. Alternatively, the RF processor 42 may include a complex demodulator (not shown) that demodulates the RF signal to form IQ data pairs representative of the echo signals. The I and Q values of the beams represent in-phase and quadrature components of a magnitude of echo signals reflected from a point P at the range R and the angle θ (shown in FIG. 6). The RF or IQ signal data may then be routed directly to a RF/IQ buffer 44 for temporary storage. A signal processor 46 may compute the magnitude (I²+Q²)^{1/2}. In an alternative embodiment, multiple filters and detectors are used so that beams received by the filters and detectors are separated into multiple passbands that are individually detected and recombined to reduce speckle by frequency compounding.

The signal processor 46 generally processes the acquired ultrasound information (i.e., RF signal data or IQ data pairs) and prepares frames of ultrasound information for display on a display system 48. The signal processor 46 is adapted to perform one or more processing operations (e.g., compounding) according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 44 during a scanning session and processed in less than real-time in a live or off-line operation.

The ultrasound system 30 may continuously acquire ultrasound information at a frame rate that exceeds fifty frames per second, which is the approximate perception rate of the human eye. The acquired ultrasound information is displayed on the display system 48 at a slower frame-rate. An image buffer 50 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 50 may comprise any known data storage medium.

The transducer elements 34 are driven such that the ultrasonic energy produced is directed, or steered, in a beam. To accomplish this, respective transmit focus time delays (not shown) are imparted to a respective transducer element 34 via Transmit/Receive (T/R) switches (not shown). As an example, transmit focus time delays may be read from a look-up table. By appropriately adjusting transmit focus time delays, the steered beam can be directed away from a y-axis by an angle θ or focused at a fixed range R on a point P.

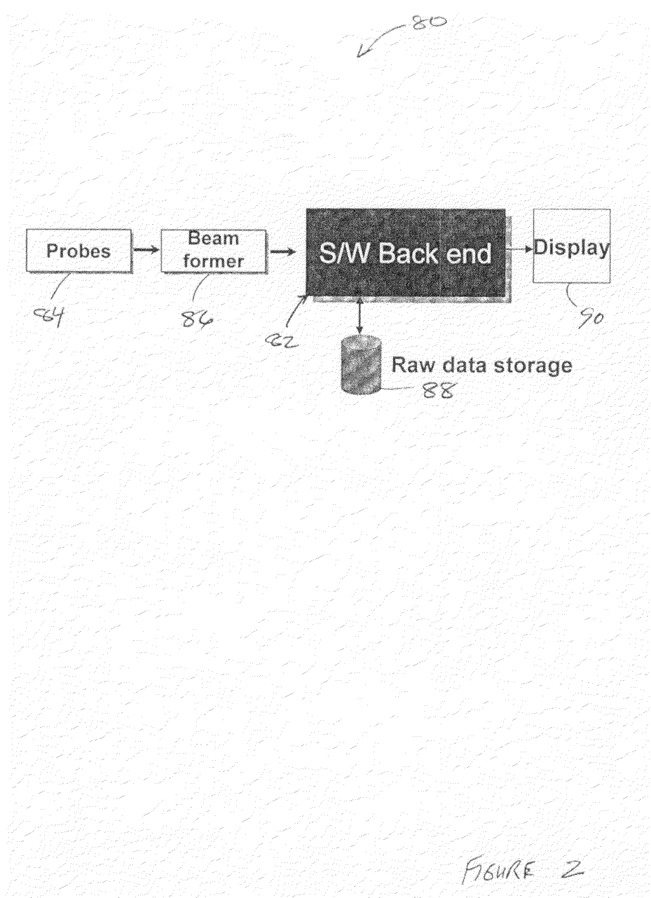

FIG. 2 illustrates a handheld or hand-carried ultrasound system 80 that utilizes a software backend 82 in accordance with various embodiments of the present invention. Ultrasound system 80 includes a probe or transducers 84, a beamformer 86, the software backend 82, a raw data storage 88 and a display 90. The raw data storage 88 may be an image buffer or, alternatively, may be a non-volatile memory element, such as a flashcard (e.g., 5 GB memory capacity) or a hard-drive. The display 90 may be configured to display at one or more different resolutions. For example, the screen may be a 160×160 screen, a 240×240 screen, a 320×480 screen, a 1024×768 screen, among others and combinations thereof. The display 90 may be configured for grayscale display, for example, display of at least 256 shades of gray scale, or may optionally display in color, for example, at least 65,000 colors. The display 90 may be configured as a national television system committee (NTSC) standard display or may be configured as a phase-alternating line (PAL) display, among others.

Typical ultrasound systems include a mid-processor, a scan converter and a host computer (not shown) between the beamformer 86 and display 90. In various embodiments, the software backend 82 replaces the mid-processor, scan converter, and host computer and, thus, performs the typically hardware intensive functions. The software backend 82 alternatively may be implemented in one of more dedicated hardware components, for example, an application specific integrated circuit (ASIC), a digital signal processor (DSP), a Field Programmable Gate Array (FPGA), and the like as described in more detail below.

The software backend 82 may include one or more modules or, if hardware implemented, processing elements. For example, the software backend 82 may include one or more modules, such as a data capture module, a geometric transformation module, an interpolation module, a compounding module, a battery management module, a heat management module, a resolution selection module, a scan conversion module and a frame processing module, among others. The geometric transformation module translates an acquisition coordinate space, either in polar or Cartesian coordinates, to a Cartesian display space. The interpolation module performs interpolation, for example, bi-linear interpolation or tri-linear interpolation. The compounding module combines a plurality of steering frames corresponding to a plurality of different angles to produce a compound image. The compounding module also controls steering of a plurality of transducer elements to a multiple angles, and may control the steering of the plurality of transducer elements to a plurality of pre-set angles. The battery management module manages and/or controls the power level of a power source, regulates current and voltage, displays battery capacity to a user, controls charging of the battery, and performs other power related functions, such as saves data to a memory when battery voltage drops below an internal low-voltage threshold. The heat management module controls heat dissipation within the device, for example, by shutting off unnecessary components or excessively hot components. The frame processing module performs temporal and spatial filtering and zooms or enlarges on an image. The scan conversion module performs scan conversion on acquired image data to allow the image data to be displayed as an image. The resolution selection module controls the resolution of the displayed image, and which may include weighting multiple steering frames with different weight factors. The software backend 82 allows the hardware architecture of an ultrasound system 80 to be miniaturized and permits the migration of features found in larger ultrasound systems.

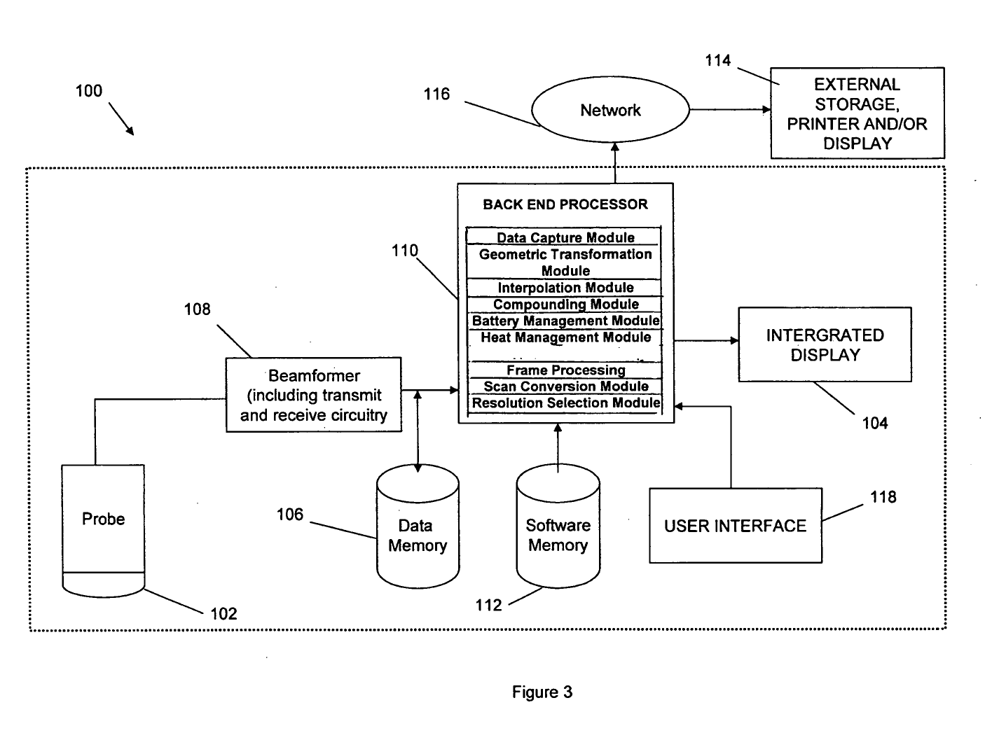

FIG. 3 is a schematic block diagram of a hand-carried or handheld medical imaging device 100 having a probe 102 or transducer configured to acquire raw medical image data in accordance with various embodiments of the invention. In some embodiments, the probe 102 is an ultrasound transducer and the hand-carried medical imaging device 100 is an ultrasound imaging apparatus. An integrated display (e.g., an internal display) 104 is also provided and is configured to display a medical image. A data memory 106 stores acquired raw image data, which may be processed by a beamformer 108 in some embodiments of the present invention. The beamformer 108 may include a transmit beamformer and a receive beamformer, which may be provided separately and in the same or different portions of the system. The transmit beamformer may utilize miniaturized components, for example, ASICs to focus the ultrasound beam or wave and angles of the beam. The receive beamformer may utilize a digital ASIC processor that includes, for example, at least 128 elements and may function to sum the reflected waves or echoes into a plurality of frames. The data memory 106 also may store one or more lookup tables that are used in an interpolation process. The hand-carried or handheld medical imaging device 100 may define a processing unit to process received scan data and perform image compounding as described in more detail herein.

To display a medical image using the probe 102, a backend processor 110 (which may implement the software backend 82 or be embodied as the software backend 82) is provided with software or firmware memory 112 containing instructions to perform, for example, data capture, geometric transformation, interpolation, compounding, battery management, heat management, frame processing, scan conversion, and resolution selection using acquired raw medical image data from probe 102. Each of these operations may be performed, for example, as part of or by separate modules. The raw medical image data also may be further processed by the beamformer 108 in some embodiments. The backend processor 110 may be an ASIC, a DSP, or a hardware processor board, such as an ETX® board commercially available from Kontron America, Poway, Calif. The processor 110 may be embedded, for example, with different operating platforms, such as Microsoft Windows® XP, Microsoft Windows® XP embedded, or Linux® software. The software or firmware memory 112 can include, for example, a read only memory (ROM), random access memory (RAM), a miniature hard drive, a flash memory card, or any kind of device (or devices) configured to read instructions from a machine-readable medium or media. The instructions contained in software or firmware memory 112 further include instructions to produce one or more medical images of suitable resolution for display on an integrated display 104, and optionally to send acquired raw image data stored in a data memory 106 to an external device 114 (e.g., higher resolution display, workstation, laptop, printer, etc.). The image data may be sent from the backend processor 110 to the external device 114 via a wired or wireless network (or direct connection) 116 under control of the back end processor 110 and a user interface 118. The wireless network 116 may be used, for example, to interface with a hospital's local area network to provide images to a physician in real-time.

The user interface 118 (which may also include the integrated display 104) is provided to receive commands from a user and to instruct the backend processor 110 to display on the integrated display 104 an image formed from the raw image data, send the acquired raw image data to the external device 114, or both, in accordance with the commands from the user.

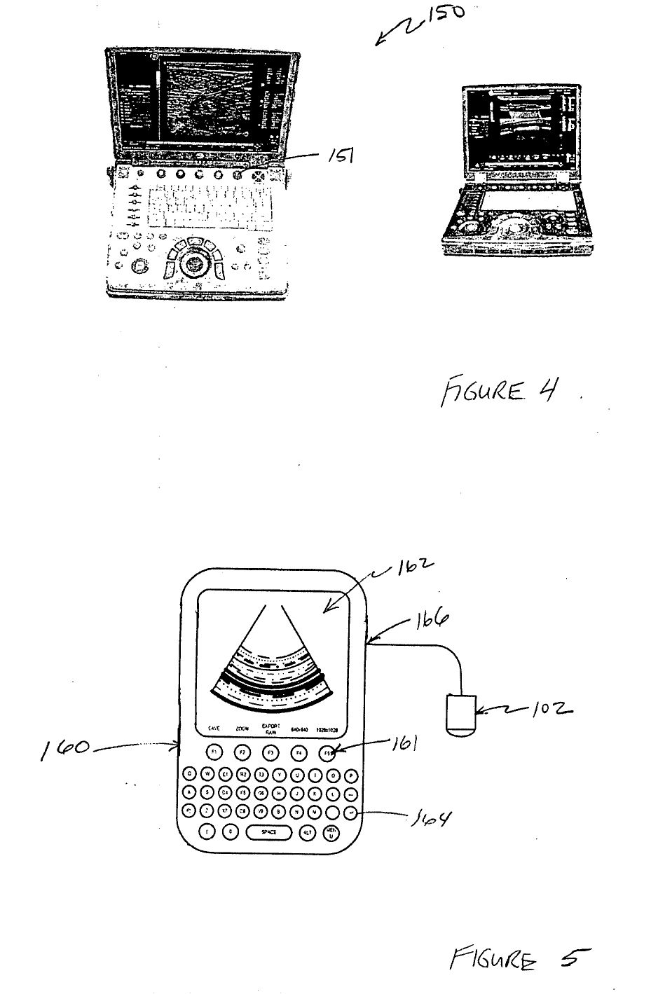

FIG. 4 illustrates a miniaturized ultrasound system 150 formed in accordance with an embodiment of the invention. As used herein, “miniaturized” means that the ultrasound system is a handheld or hand-carried device or is configured to be carried in a person's hand. For example, the ultrasound system 150 may be a hand-carried device having a size of a typical laptop computer, for instance, having dimensions of approximately 2.5 inches in depth, approximately 14 inches in width, and approximately 12 inches in height. The ultrasound system 150 may weigh about ten pounds and have a power consumption of about fifty watts. Examples of commercially available ultrasound systems in connection with which various embodiments may be implemented include, for example, the LOGIQ®e and LOGIQ®i systems, available from GE Healthcare of Waukesha, Wis.

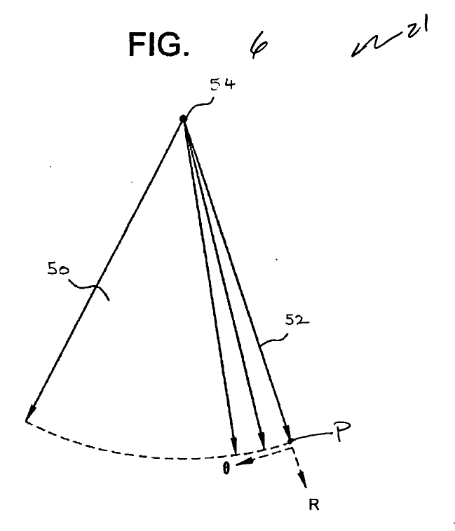

Alternatively, the ultrasound system 150 may be a handheld device and fit in the palm of a user's hand and be approximately 2.5 inches wide, approximately 4.0 inches in length, and approximately 0.5 inches in depth and weighing between about 7 and about 16 ounces. Optionally, the ultrasound system 150 may be approximately 3.1 inches wide, approximately 4.75 inches in length, and approximately 1 inch in depth. Yet, another alternative is for the ultrasound system 150 to be configured to fit in a person's pocket. FIG. 5 shows an exemplary example of a pocket-sized ultrasound system 160. The pocket sized device may be approximately 2 inches wide, approximately 4 inches in length, and approximately 0.5 inches in depth and weigh less than 3 ounces. The pocket-sized ultrasound system 160 generally includes a display 162, a user interface 164 (e.g., keyboard) and an input/output (I/O) port 166 for connection to a probe, for example, the probe 102. However, it should be noted that the various embodiments may be implemented in connection with a miniaturized ultrasound system having different dimensions, weights, and power consumption. For example, power consumption may be in the order of seven to ten watts.

The various embodiments may perform scanning as shown in FIG. 6 that illustrates a sector scan 21 that is performed by scanning a fan-shaped two-dimensional (2D) region 50. The sector scan 21 scans the region 50 along a direction of the angle θ and along an acoustic line 52 extending from an emission point 54.

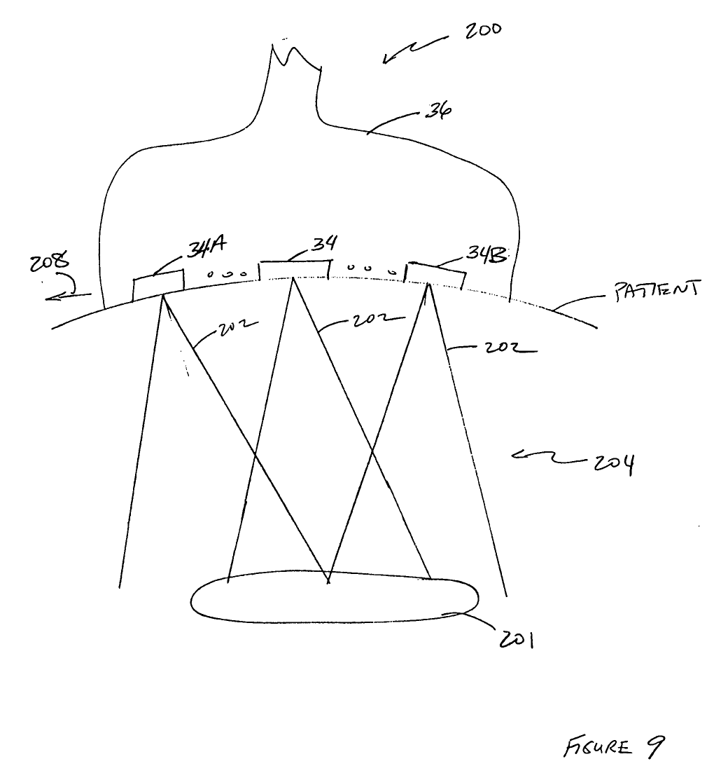

FIG. 7 alternatively illustrates a linear scan that is performed by scanning a rectangular 2D region 60 in a direction along an x-axis. The rectangular region 60 is scanned in a direction along the x-axis by translating acoustic line 52, which travels from emission point 54 in a direction along the y-axis.

FIG. 8 illustrates a convex scan or a curved linear scan that is performed by scanning a partial fan-shaped region 70 in the direction of the angle θ. Partial fan-shaped region 70 is scanned in the direction of the angle θ by performing an acoustic line scan similar to the linear scan and moving emission point 54 of acoustic line 52 along an arc-shaped trajectory 72.

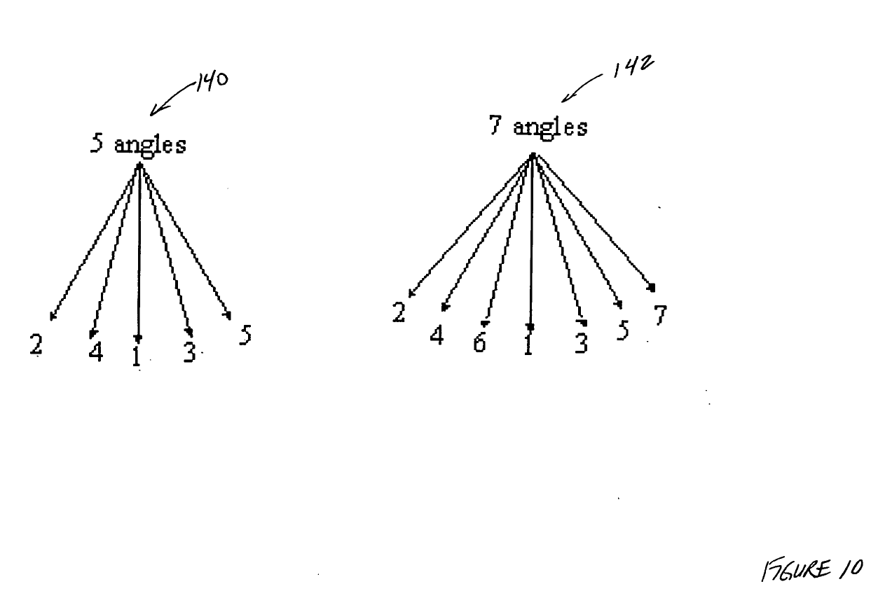

FIG. 9 illustrates an exemplary acquisition 200 of an object 201 acquired by system 30 (shown in FIG. 1) in accordance with an embodiment of the invention. Transducer 36 includes a plurality of transducer elements 34 (e.g., an array of piezoelectric elements) positioned linearly along an edge of the transducer 36. The transducer 36 is typically in contact with a patient's skin. The transducer elements 34 are coupled to transmitter 32 and receiver 38 (all shown in FIG. 1) and are responsive to transmit signals from transmitter 32 to generate an ultrasound beam or wave 202 that emanates from the edge of array transducer 36 proximate to each transducer element 34. The transmit signals may be phased to control the firing of each transducer element 34 to steer ultrasound wave 202 along a predetermined path (e.g., a parallel path toward object 201). For illustration purposes only, four transducer elements 34 are illustrated. The transducer 36 may include any number of transducer elements 34. Each wave 202 is projected into a volume of interest 204 that may contain an object of interest 201 and may overlap one or more of waves 202 emanating from adjacent transducer elements 34. Object 201 may absorb, transmit, refract and/or reflect waves 202 that impact object 201. Reflected waves or echoes from object 201 are received by transducer elements 34 and processed by system 30 to create image or steering frames indicative of the object 201 and other objects within volume 204.

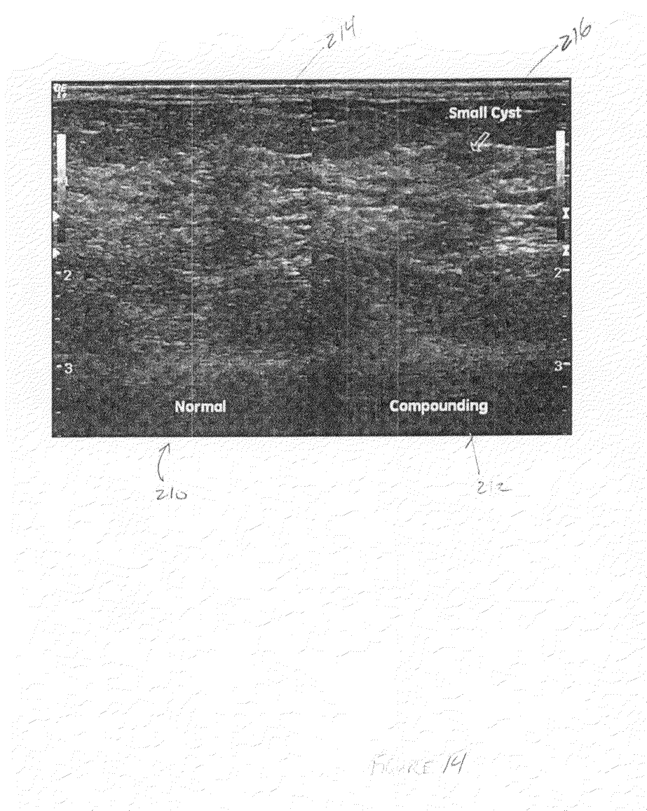

Transducer elements 34 may be steered at different angles to transmit 32 or receive 38 the ultrasound beam or wave 202. Generally, there are three categories of steering: a “no steer” direction 130, a “left steer” direction 134 and a “right steer” direction 136 (as shown in FIG. 13). Steering in the various embodiments is accomplished electronically using programming delays in the firing sequence of the transducer elements 34. In the no steer direction 130, the transducer elements 34 are controlled (e.g., selectively activated) to transmit the ultrasound beam or wave 202 in a parallel line that is perpendicular (e.g., at approximately 90 degrees) to a ROI 204. The receiver 38 then receives a plurality of echoes from the ROI 204 that are combined by receiver beamformer 40 into a no steer frame.

Alternatively, the transducer elements 34 may be steered to transmit the ultrasound beam or wave 202 at different angles, for example, to the left or right of a parallel line that is perpendicular (e.g., at approximately 90 degrees) to a ROI 204. For example all the transducer elements 34 may be controlled to transmit the ultrasound beam or wave 202 at one particular angle. On the other hand, a group of transducer elements 34A (shown in FIG. 9) may be steered to a “left steer” direction 134 (as shown in FIG. 13), where the ultrasound beam or wave 202 is transmitted at an obtuse angle (e.g., between 90-180 degrees). Another group of transducer elements 34B (shown in FIG. 9) may be steered at a “right steer” direction 136, where the ultrasound beam or wave 202 is transmitted at an acute angle (e.g., between 0-90 degrees).

In the various embodiments, the transducer elements 34 may be steered by providing different delays to different transducer elements 34 in a transmit aperture. Each ultrasound beam or wave 202 is transmitted using an aperture of a plurality of transducer elements 34. The delay between different transducer elements 34 defines the steering and focus direction of the ultrasound beam 202. The transducer elements 34 include piezoelectric elements (not shown) that fire a short ultrasound pulse. By using different time belays between the firing of piezoelectric elements in the aperture, a transmit beam may be converged or steered. For example, to steer to the right, the transducer elements 34 on the left side of the aperture are fired first with no delay or a short delay, and the elements on the right side of the aperture fired last with increasing longer delays. Thus, the ultrasound beam or wave 202 would converge or focus at an angle steered to the right.

After the transmission of the ultrasound beam or wave 202 from either a right steer direction 134 or a left steer direction 136, the receiver 38, then receives a plurality of echoes from the ROI 204 that are combined by receiver beam former 40 into either a right steer frame or a left steer frame corresponding to the transmitted wave 202.

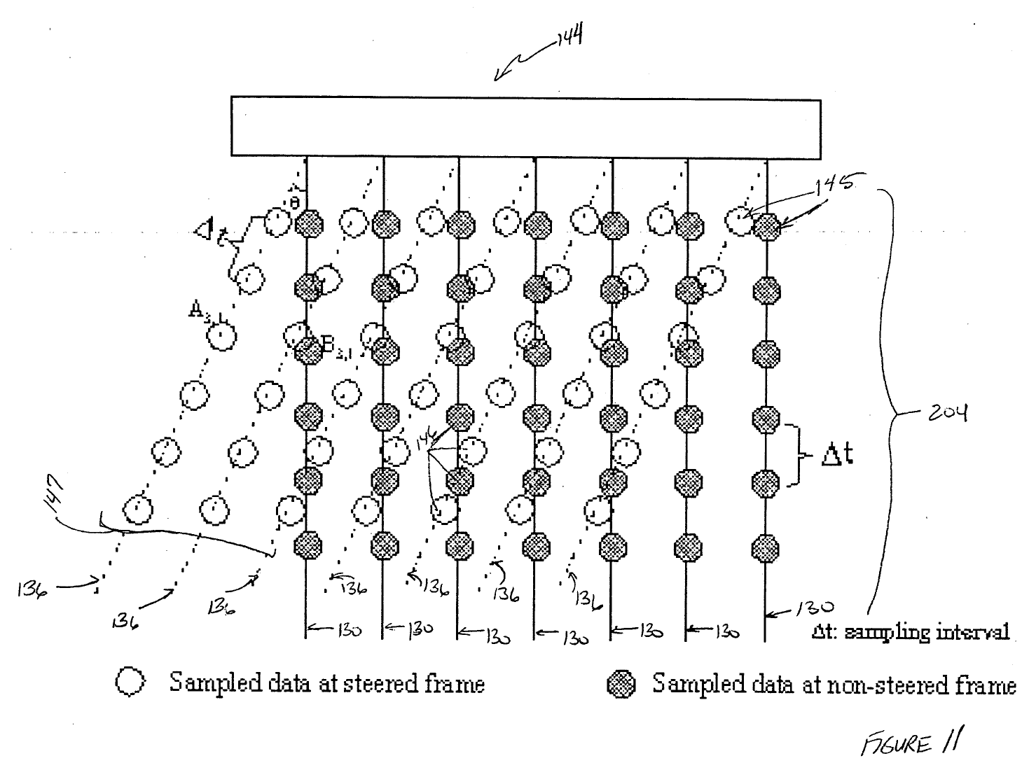

FIG. 10 illustrates two sequences 140 and 142 for steering the transducer elements 34 in accordance with an embodiment of the invention. The first sequence 140 depicts five steer directions that the transducer elements 34 may be steered to prior to transmission of the ultrasound beam or wave 202. The transducer elements 34 remain in the steered direction through at least one transmit 32 and receive 38 cycle. The ultrasound beam or wave 202 is transmitted using an aperture of a plurality of transducer elements 34. Initially, the transducer elements 34 are provided in a “no steer” direction 130, corresponding to the number one. The transducer elements 34 are then provided in a right steer direction two (2), followed by a left steer direction three (3), then back to a right steer direction four (4) then to a left steer direction five (5), and finally returning to the no steer direction one (1) and the process repeats. Similarly, sequence 142 shows seven angle directions to which the transducer elements 34 are steered prior to transmission of the ultrasound beam or wave 202. In an embodiment, the firing of the transducer elements 34 is sequentially changed in about 100 micro-second intervals.

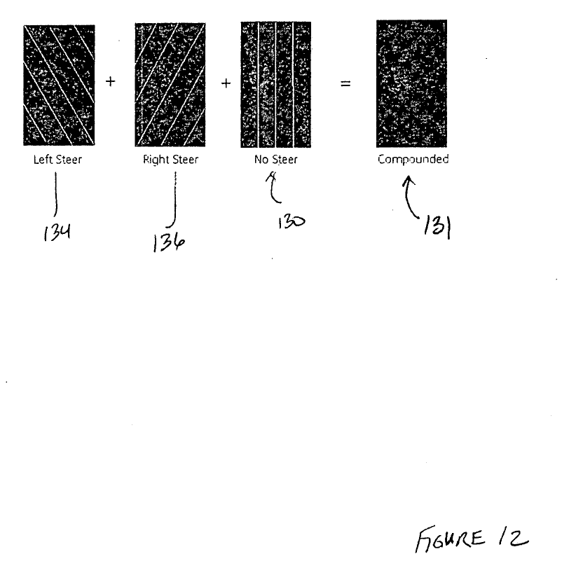

FIG. 11 illustrates the acquisition 144 of data samples 145 from a steered frame 136 and a non-steered frame 130 in accordance with an embodiment of the invention. The steered frame 136 is shown as a right steer 136, however, a left steer 134 may be used. The sampling interval 145 for both steered frame 136 and non-steered frame 130 is constant. As shown, by using a combination of steered frames 136 and non-steered frames 130, greater resolution of a region of interest is possible by acquiring multiple samples 146 co-located near one another. In addition, a greater coverage area 147 is possible by acquiring samples that are angled away from a parallel line that is perpendicular to a ROI 204.

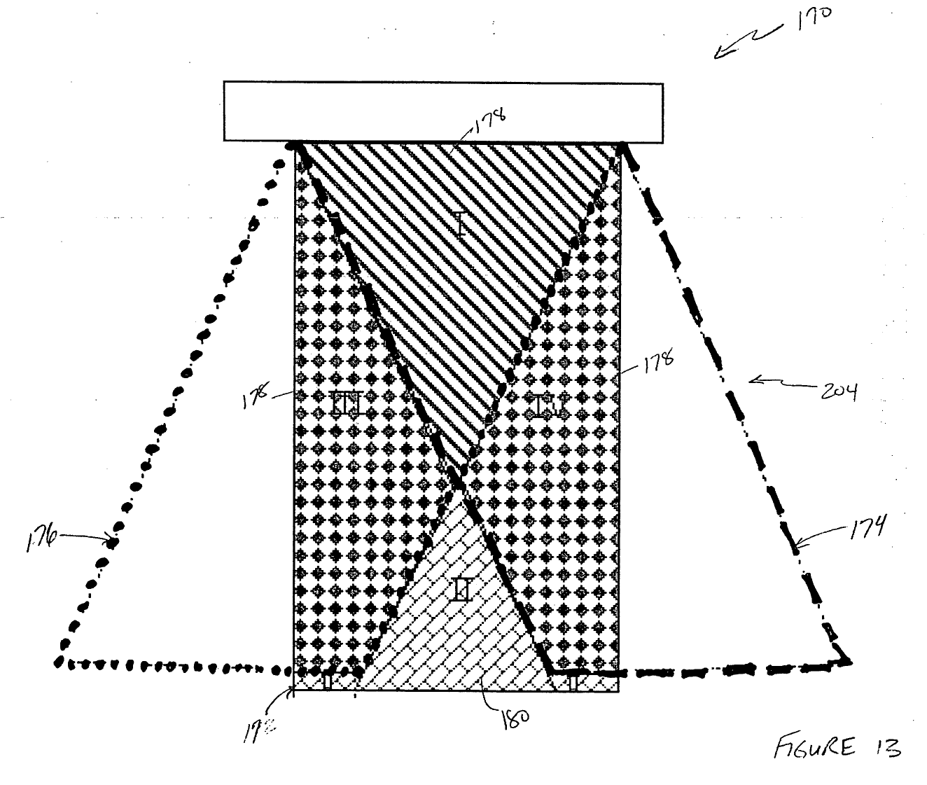

FIG. 12 is a schematic illustration of spatial compounding in accordance with an embodiment of the invention. Spatial compounding is an imaging technique in which a number of echo signals from a number of multiple look directions or angles are combined. The multiple directions help achieve speckle decorrelation. FIG. 12 shows an example of three steering frames. The steering frames correspond to a set of received echoes based on the steering of transducer elements 34 when transmitting the ultrasound beam or wave 202 in parallel as well as at different angles. A left steering frame 134, a right steering frame 136 and a no steer frame 130 are combined to produce a compounded image 131.

FIG. 13 illustrates a weighting factor for three-angle compounding 170 in accordance with an embodiment of the invention. As an example of compounding, three different angles are used to acquire scan data. A first angle 172 (shown as the area between the solid lines) corresponds to a no steer direction 130. A second angle 174 (shown as the area between the dashed lines) corresponds to a left steer direction 134, and a third angle 176 (shown as the area between the dotted lines) corresponds to a right steer direction 136. Three overlap areas 178 are depicted as area I, area III and area IV. A non-over lap area 180 is shown as area II. Relative weights are assigned to the three areas prior to combining them to produce a compound image 131 (as shown in FIG. 12). For instance, the overlap areas 178 may be assigned the same weighting factor. Alternatively, different weights may be assigned to each of the areas I, II, III and IV. By weighting the acquired scan data differently, speckle interference may be decreased, thereby improving image quality. In addition, weighting eliminates any detected motion prior to combining the plurality of steering frames into a compound image. Alternatively, different levels of compounding may be used. For instance, a high level of compounding (e.g., five frame images, transducer elements 34 steered at large angles) may be used, for example, when regional anesthesia is applied to a patient. Other applications may require no compounding or a lower level of compounding (e.g., three frame images and transducer elements 34 steered at smaller angles). Therefore, in an embodiment, a plurality of default preset choices for different levels of compounding may be provided (e.g., no compounding, low compounding, high compounding). The preset compounding choices may also be programmed on soft keys 151 (shown in FIG. 4) and 161 (shown in FIG. 5) in a hand-carried or handheld device, respectively.

A predetermined number of image frames are then combined into a compound image by the ultrasound system 30. The compound image may include frames representative of views of object 201 from different angles enabled by the spatial separation of transducer elements 34 along array transducer 36. For instance, frames representing a left steering frame, a right steering frame, and a no steering frame are combined to produce a compound image. Errors in angle due to refraction may cause misregistration between frames that view object 201 from different angles. Misregistration between the image frames may also occur due to motion 208 of array transducer 36 during the transmit and receive process. Image frames may be separated from each other in time as well as spatially.

Misregistration between steering frames can be measured by a number of motion tracking methods such as a correlation block search, Doppler tissue velocity, accelerometers or other motion sensors, and feature tracking. The degree of misregistration may also be detected by a cross correlation method. Alternatively, motion 208 of array transducer 36 may also be detected by comparing the information of compounded images. Operating the ultrasound system 30 in various modes is selectable by the user. In an exemplary embodiment, the handheld medical imaging device 100 determines an optimum number of frames to be used in constructing the compounded image automatically and continuously. In an alternative embodiment, the user may select the number of frames used to construct the compound image manually.

The ultrasound system 30 detects 208 motion of array transducer 36 and also detects a rate of change of motion of the transducer 36. The motion and rate of change of motion signals are compared to predetermined limit values to modify the image process of the ultrasound system 30. Specifically, the motion of the transducer 36 may be used to determine a number of image frames that is used in constructing a compound image. The rate of change of motion of the transducer 36 may be used to determine a delay period before the number of image frames used to construct the compound image is modified based on motion of the transducer 36. Additionally, the rate of change of motion of the transducer 36 may be used to determine the number of image frames used to construct the compound image directly. After the motion of the transducer 36 is determined the ultrasound system 30 combines a plurality of steering frames into a compound image based on the detected motion and rate of change of motion of the transducer 36.

In operation, the ultrasound system 30 may use a first number of frame images to construct a compound image (e.g., three or five frame images), when the transducer 36 is maintained substantially stationary with respect to the body being scanned. If the transducer 36 is placed into motion 208 with respect to the body, the ultrasound system 30 detects the motion 208 and the rate of change of the motion of the transducer 36. If motion 208 of the transducer 36 exceeds a predetermined value, the ultrasound system 30 may modify the number of image frames used to construct a compound image to reduce the effects of motion 208. The ultrasound system 30 may incorporate a delay, such that the number of frames used to construct the compound image is not modified immediately upon the transducer 36 exceeding the predetermined value. The delay may be useful to maintain display image stability during periods when the transducer 36 may be moved a relatively short distance or for a relatively short period of time. It may be the case though, that rapid motion of the transducer 36 may be detrimental to display image stability. For example, a relatively large increase the rate of change of motion of the transducer 36 may indicate the misregistration of the upcoming image frames will be large, such that a compound image constructed from the current number of image frames may be unusable due to poor image stability. Based on the rate of change of motion of the transducer 36, the ultrasound system 30 may modify the number of frame images used to construct a compound image to a second number of frame images that facilitates maintaining stability of the displayed image. The ultrasound system 30 may modify the time delay used between when the rate of change of motion of the transducer 36 is detected to be exceeding a predetermined value and when the ultrasound system 30 modifies the number of frame images used to construct the compound image.

In addition, the ultrasound system 30 provides for reducing interference caused by speckle noise. Speckle noise is an intrinsic property of ultrasound imaging, the existence of speckle noise in ultrasound imaging reduces image contrast and resolution. A speckle reduction filter is used to reduce speckle noise. The speckle reduction filter usually does not create motion artifacts, preserves acoustic shadowing, and enhancement. However, the speckle reduction filter may cause a loss of spatial resolution and reduce processing power of an ultrasound imaging system.

A speckle reduction filter (not shown), such as a low pass filter, may be utilized to reduce speckle noise in an image generated the ultrasound system 30. An example of a low pass filter is a finite impulse response (FIR) filter. In an alternative embodiment, the speckle reduction filter is a mathematical algorithm that is executed by the processor 36 and that is used on a single image frame to identify and reduce speckle noise content. In yet another embodiment, the speckle reduction filter is a median filter, a Wiener filter, an anisotropic diffusion filter, or a wavelet transformation filter, which are mathematical algorithms executed by the processor 36. In still another alternative embodiment, the speckle reduction filter is a high pass filter that performs structural and feature enhancement. An example of a high pass filter is an infinite impulse response (IIR) filter. In the median filter, a pixel value of an image generated using the ultrasound system 30 is replaced by a median value of neighboring pixels. The Wiener filter can be implemented using a least mean square (LMS) algorithm. The anisotropic diffusion filter uses heat diffusion equation and finite elements schemes. The wavelet transformation filter decomposes echo signals into a wavelet domain and obtained wavelet coefficients are soft-thresholded. In the soft-thresholding, wavelets with absolute values below a certain threshold are replaced by zero, while those above the threshold are modified by shrinking them towards zero. A modification of the soft thresholding is to apply nonlinear soft thresholding within finer levels of scales to suppress speckle noise.

It should be noted that the systems and methods for implementing a speckle reduction filter can be used in conjunction with a computer-aided diagnosis (CAD) algorithm. As an example, the CAD algorithm is used to distinguish different organs, such as liver and kidney. As another example, the CAD algorithm is used to distinguish liver cancer from normal tissues of the liver. The CAD algorithm can be implemented for real time imaging or for imaging that is to be performed at a later time.

Another technique to reduce speckle noise is compounding that may be used in conjunction with a speckle reduction filter. Compounding includes spatial compounding and frequency compounding. Frequency compounding and spatial compounding, which are described below, have been explored as ways to reduce the speckle noise. However, frequency and spatial compounding have limitations of slower frame rate, motion artifacts, or reduced resolutions. Image processing filters are alternatives to compounding. The image processing filters operate on image data instead of front-end acquisitions, and they usually do not have problems, such as loss of frame rate or loss of acoustic shadow, associated with compounding.

Spatial compounding is an imaging technique in which a number of echo signals of the point P (as shown in FIG. 2) that have been obtained from a number of multiple look directions or angles are combined. The multiple directions help achieve speckle decorrelation. For frequency compounding, speckle decorrelation is achieved by imaging the point P with different frequency ranges. The frequency compounding is performed in a B-mode processor (not shown) or a Doppler processor (not shown). Similarly, the spatial compounding is performed in B-mode processor or the Doppler processor. By combining spatial compounding with the methods for implementing a speckle reduction filter, the number of angles can be reduced, for instance, from nine to three, to reduce motion artifacts while maintaining a level of speckle noise reduction. However, alternatively, the spatial or the frequency compounding may not be performed.

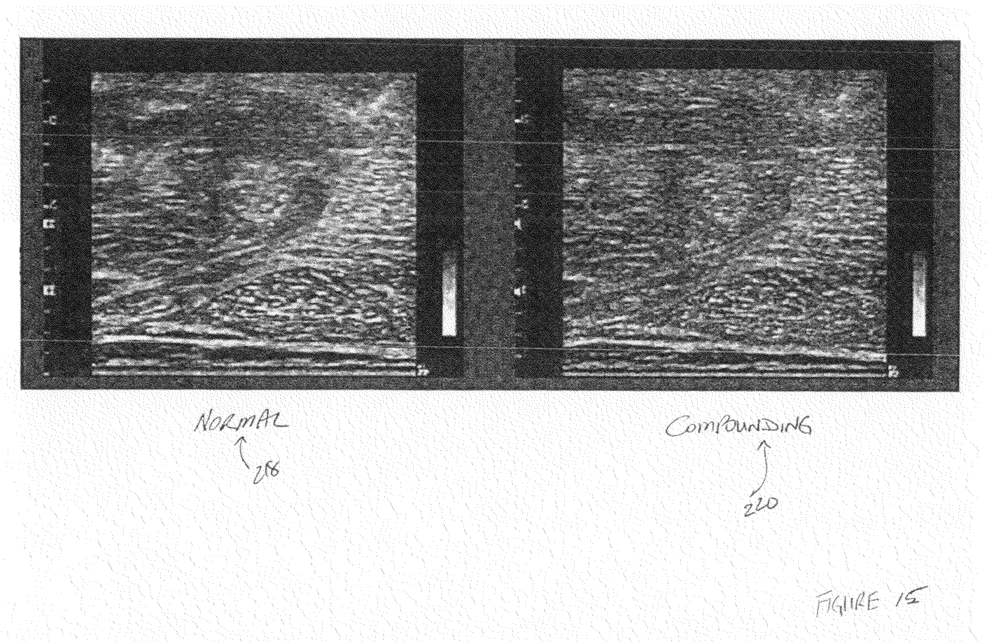

FIGS. 14 and 15 illustrate a normal ultra-sound image and an ultra-sound image using spatial compounding in accordance with an embodiment of the present invention. In an embodiment, a simultaneous view of a spatial compounded view and a non-compounded view is displayed. FIG. 14 shows a comparison of an image 210 obtained by a normal ultrasound techniques compared to an image 212 obtained by spatial compounding. As shown in the normal image 210, typically, multiple parallel scan lines are directed directly towards, for example, a tendon 214. The multiple parallel scan lines result in image 210 of the tendon 222. However the image 210 fails to show any structures beneath tendon 214 that are hidden from view. As shown in image 212, by permitting each transducer element 34 (shown in FIG. 1) to be independently steered at multiple angles, non-perpendicular scan lines are generated that provide better imaging of structures hidden beneath other objects (e.g., a needle). As shown in image 212, by directing the ultrasound beam or wave 202 at multiple angles around the tendon 214 combined with spatial compounding allows a cyst 220 located beneath tendon 214 to be imaged.

FIG. 15 further illustrates a normal image 218 and a spatial compounding image 220 that compare anatomical structures with diagonal borders in accordance with an embodiment of this invention. Normal image 218 is acquired by having a user incline the transducer 36 laterally with the transducer 36 at a different steering angle while maintaining the transducer substantially in the same position. Therefore there is an angular dependence associated with the transducer 36 to acquire the normal image 218. The spatial compounding image 220 eliminates the angular dependence of the transducer 36 by using multiple angled scan lines. The multiple angled scan lines in combination with spatial compounding allows the visualization of continuous boundaries and interfaces. In addition, by using multiple angled scan lines combined with spatial compounding reduces speckle and provides better image resolution.

A technical effect of the various embodiments is to use a handheld ultrasound device or handheld ultrasound system to provide better imaging of structures hidden beneath other objects, showing continuous boundaries and interfaces between anatomical structures, less angular dependence when viewing anatomical structures with diagonal or vertical borders, and decreasing the number of speckles by using spatial compounding and images obtained multiple angles.

The various embodiments or components thereof may be implemented as part of a computer system. The computer system may include a computer, an input device, a display unit, and an interface, for example, for accessing the Internet. The microprocessor may be connected to a communication bus. The computer may also include a memory. The memory may include Random Access Memory (RAM) and Read Only Memory (ROM). The computer system further may include a storage device, which may be a hard disk drive or a removable storage drive such as a floppy disk drive, optical disk drive, and the like. The storage device can also be other similar means for loading computer programs or other instructions into the computer system.

In various embodiments of the invention, the method of forming an ultrasound image as described herein or any of its components may be embodied in the form of a processing machine. Typical examples of a processing machine include a general-purpose computer, a programmed microprocessor, a digital signal processor (DSP), a micro-controller, a peripheral integrated circuit element, and other devices or arrangements of devices, which are capable of implementing the steps that constitute the methods described herein.

As used herein, the term “processor” may include any computer, processor-based, or microprocessor-based system including systems using microcontrollers, reduced instruction set circuits (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The processing machine executes a set of instructions (e.g., corresponding to the method steps described herein) that are stored in one or more storage elements (also referred to as computer usable medium). The storage element may be in the form of a database or a physical memory element present in the processing machine. The storage elements may also hold data or other information as desired or needed. The physical memory can be, for example, but not limited to, an electronic, magnetic, optical, electromagnetic, infrared, or semiconductor system, apparatus, device, or propagation medium. More specific examples of the physical memory include, but are not limited to, the following: a random access memory (RAM) a read-only memory (ROM), an erasable programmable read-only memory (EPROM or Flash memory), a Hard Disc Drive (HDD) and a compact disc read-only memory (CDROM).

The set of instructions may include various commands that instruct the processing machine to perform specific operations such as the processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

In various embodiments of the invention, the method of creating an ultrasound medical image can be implemented in software, hardware, or a combination thereof. The methods provided by various embodiments of the present invention, for example, can be implemented in software by using standard programming languages such as, for example, C, C++, Java, and the like.

As used herein, the terms “software” and “firmware” are interchangeable, and include any computer program stored in memory for execution by a computer, including RAM memory, ROM memory, EPROM memory, EEPROM memory, and non-volative RAM (NVRAM) memory. The above memory types are exemplary only, and are thus limiting as to the types of memory usable for storage of a computer program.

The analysis described above may be performed on several different data sets. Calculations may be performed on individual slices or rings or detectors, groups of slices, all slices, or a select line of responses, specific r and θ ranges, and the like. The analyzed data set may be modified to focus on the motion of specific organs or structures. The physiological structure may include a biological organ, for example, the stomach, heart, lung or liver; a biological structure, for example, the diaphragm, chest wall, rib cage, rib, spine, sternum or pelvis; or a foreign object fiducial marker, for example, a marker placed for the purpose of gating; a tumor, or a lesion or sore, for example, a bone compression fracture.

Thus, a handheld ultrasound device or handheld ultrasound system is provided that uses spatial compounding to provide better imaging of structures hidden beneath other objects, shows continuous boundaries and interfaces between anatomical structures and less angular dependence when viewing anatomical structures with diagonal or vertical borders and has a decreased number of speckles because speckles obtained from different angles are incoherent.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

While the invention has been described in terms of various specific embodiments, those skilled in the art will recognize that the invention can be practiced with modification within the spirit and scope of the claims.

What is claimed is:

1. An ultrasound system, comprising:
a probe configured to acquire scan data; and
a processing unit configured to process the received scan data and perform image compounding, the processing unit being one of a handheld device and a hand-carried device.

2. The system according to claim 1, wherein the probe is one of an ultrasonic probe and an ultrasound transducer.

3. The system according to claim 1, wherein the probe comprises a plurality of transducer elements in a transducer array, the transducer elements are programmed to be steered at a plurality of different angles.

4. The system according to claim 3, wherein the transducer elements are steered to align to at least a right side or a left side of a parallel line, the parallel line perpendicular to a region of interest.

5. The system according to claim 4, wherein a first group of transducer elements are in a no steer direction, a second group of transducer elements are in a right steer direction, and a third group of transducer elements are in a left steer direction.

6. The system according to claim 3, wherein the plurality of different angles comprise at least one of a left steer direction, a right steer direction and a no steer direction.

7. The system according to claim 1, wherein the probe transmits a plurality of ultrasound waves at a plurality of different angles to a region of interest.

8. The system according to claim 1, wherein the probe receives ultrasound echoes for a plurality of transmitted ultrasound waves, each set of received echoes define a plurality of steering frames corresponding to a plurality of different angles.

9. The system according to claim 1, wherein the processing unit comprises a backend processor, the backend processor combining a plurality of steering frames to produce a compound image.

10. The system according to claim 9, wherein the backend processor is configured to send acquired raw image data to an external device using one of a wired and wireless network.

11. The system according to claim 1, wherein the processing unit is configured to process raw image data, the processor unit further comprising at least one of a data capture module, a geometric transformation module, an interpolation module, a compounding module, a battery management module, a heat management module, a frame processing module, a scan conversion module, and a resolution selection module.

12. The system according to claim 11, wherein the battery management module controls one of the power level of a battery, regulates current and voltage, displays battery capacity to a user, controls charging of the battery, and saves data to a memory when battery voltage drops below an internal low-voltage threshold.

13. The system according to claim 11, wherein the heat management module controls heat dissipation.

14. The system according to claim 1, further comprising software memory with instructions configured to control the processing unit, the software memory comprising a non-volatile memory card.

15. The system according to claim 1, wherein the image compounding comprises combining at least a left steering frame, a right steering frame and a no steering frame and combinations thereof to produce a compound image.

16. The system according to claim 1, wherein the image compounding comprises weighting a left steering frame, a right steering frame, and a no steering frame such that the weighting eliminates any detected motion prior to combining the plurality of steering frames into a compound image.

17. The system according to claim 1, wherein the image compounding comprises at least one of a no compounding, a low compounding, and a high compounding, where the compounding is selected from a plurality of soft keys.

18. The system according to claim 1, further comprising a display to simultaneously display a compounded image and a non-compounded image.

19. The system according to claim 1, wherein the handheld device or the hand-carried device is configured to consume less than fifty watts of power.

20. The system according to claim 1, wherein the handheld device or the hand-carried device is configured to consume less than ten watts of power.

21. The system according to claim 1, wherein the handheld device or the hand-carried device is housed within a case and together having a total weight less than ten pounds.

22. The system according to claim 1, wherein the handheld device or hand-carried device is housed within a case and together having a total weight less than two pounds.

23. The system according to claim 1, wherein the handheld device or hand-carried device is housed within a case having a length less than about four inches and a width less than about two inches.

24. The system according to claim 1, wherein the handheld device or hand-carried device is housed within a case allowing single hand operation.

25. A method of medical ultrasound imaging using a hand-carried ultrasound imaging system that includes a transducer array, said method comprising:
transmitting a plurality of ultrasound waves at a plurality of different angles from the transducer array into a region of interest;
receiving ultrasound echoes for each of the transmitted waves, each set of received echoes defining a plurality of steering frames corresponding to the plurality of different angles; and
combining a plurality of steering frames in the hand-carried ultrasound imaging system to produce a compound image.

26. The method in accordance with claim 25, further comprising displaying a compound image and a non-compound image adjacent to one another on a screen.

27. A medical ultrasound system, comprising:
a transducer array including a plurality of transducers for transmitting ultrasound signals at a plurality of different angles into a region of interest;
a receiver for receiving ultrasound echoes for each transmitted ultrasound signal, each set of received echoes defining a plurality of steering frames corresponding to the plurality of different angles; and
a signal processor in one of a handheld device and a hand-carried device for combining said steering frames into a compound image.

28. A computer readable medium for use in a handheld or hand-carried medical ultrasound imaging system having an array transducer for transmitting and receiving ultrasound signals into a region of interest, the computer readable medium comprising:
i) instructions to transmit ultrasound signals at a plurality of different angles into the region of interest;
ii) instructions to receive ultrasound echoes for each of the transmitted ultrasound signals, wherein each set of received echoes defines a plurality of steering frames corresponding to the plurality of different angles;
iii) instructions to filter the steering frames using a speckle filter to remove interference of scattered echo signals reflected from the region of interest;
iv) instructions to combine a plurality of the filtered steering frames into a compound image; and
v) instructions to display the compound image.

29. The media of claim 28, further comprising instructions configured to instruct a backend processor to perform at least one of a data capture, a geometric transformation, an interpolation, an image compounding, battery management, heat management, frame processing, scan conversion, and resolution selection.
